(19) United States
(12) Patent Application Publication
(10) Pub. No.: US 2006/0188975 A1
(43) Pub. Date: Aug. 24, 2006

(54) Genetically modified somatic cells for sustained secretion of lysosomal proenzymes deficient in lysosomal storage disorders

(76) Inventor: Mani Ramaswami, Tucson, AZ (US)

Correspondence Address: TRASK BRITT, P.O. BOX 2550, SALT LAKE CITY, UT, 84110, US

(21) Appl. No.: 11/357,542

(22) Filed: Feb. 17, 2006

Related U.S. Application Data
(63) Continuation of application No. PCT/US04/27124, filed on Aug. 20, 2004.
(60) Provisional application No. 60/496,830, filed on Aug. 21, 2003.

(51) Int. Cl. C12N 9/36; C12N 5/08

(52) U.S. Cl. 435/206; 435/368

(57) ABSTRACT

The invention relates to various methods of treating lysosomal storage disorders using somatic cells and methods of delivering therapeutic enzymes. More particularly, gain-of-function or loss-of-function mutations in components of the intracellular Golgi to lysosome sorting pathway are used to enhance secretion of one or more lysosomal enzymes in somatic cells, thereby providing treatment for lysosomal storage disorders, particularly in neuronal cells. In addition, homologous recombination may be used to engineer therapeutically useful cells, for example, somatic cells, such as, glial progenitor cells, mesenchymal stem cells and astrocyte precursor cells, to enhance secretion of one or more lysosomal enzymes.

### CROSS-REFERENCE TO RELATED APPLICATIONS

This application is a continuation of PCT International Patent Application No. PCT/US2004/027124, filed on Aug. 20, 2004, designating the United States of America, and published, in English, as PCT International Publication No. WO 2005/021716 A2 on Mar. 10, 2005, the contents of the entirety of which is incorporated by this reference, and which claims the benefit of U.S. Provisional Application No. 60/496,830, filed Aug. 21, 2003, the contents of which are also incorporated by this reference.

### TECHNICAL FIELD

The invention relates to biotechnology generally and, more particularly, to various means and methods of treating lysosomal storage disorders using somatic cells and methods of delivering therapeutic enzymes.

### BACKGROUND

Lysosomal storage diseases are a large class of heritable disorders that affect close to 1 in 7000 live-born infants, the majority of whom develop central nervous system (CNS) disease (Sly and Vogler, 2002).

Lysosomes are the principle site of intracellular digestion, which consist of membrane-encapsulated vesicles containing hundreds of enzymes, including more than forty acid hydrolases, capable of degrading most biologically important macromolecules, as discussed in R. T. Dan et al., 1976. Each enzyme possesses the specialized task of degrading a particular class of molecule. Genetic mutations that affect any one of these degradation enzymes result in the lysosome storing and accumulating large quantities of material that they are unable to degrade, hence the term “lysosomal storage disease.” For example, Gaucher's syndrome results from a genetic defect in the production of an enzyme called glucocerebrosidase which degrades carbohydrates called glucocerebrosides, therefore, a decrease or loss of glucocerebrosidase activity results in lysosomal accumulation of glucocerebrosides.

An elaborate and carefully regulated intracellular pathway exists to ensure that lysosomal enzymes are specifically targeted to, and enriched, in lysosomes. This intracellular trafficking pathway, of which most important aspects are conserved in eukaryotic cells (yeast to human), is generally outlined. Secreted proteins, for example, growth factors, adhesion proteins and antibodies, as well as proteins destined for other intracellular locations, such as lysosomes, are translated from mRNAs in the cytoplasm but, during translation, the resulting proteins are inserted into a subcellular compartment called the endoplasmic reticulum (ER). From the ER, proteins are transferred, through carrier vesicles (spherical structures surrounded by a membrane bilayer), to the proximal (cis) compartment of the Golgi. From the cis-Golgi, proteins traffic through the Golgi the trans-Golgi network, a major sorting site in the secretory pathway. In the trans-Golgi, secreted proteins are packaged into transport vesicles that move to and fuse with the plasma membrane. In contrast, lysosomal enzymes, synthesized in inactive precursor form (proenzyme), are distinguished from secreted proteins by the presence of a signal recognized by sorting proteins (sortases) present in the trans-Golgi. Sortases target lysosomal proenzymes into transport vesicles that find and fuse selectively with organelles called late endosomes.

There are over thirty lysosomal diseases, each resulting from a deficiency of a particular lysosomal protein, usually as a result of genetic mutation. See, e.g., Cotran et al., Robbins Pathologic Basis of Disease (4th ed. 1989). A deficiency in a lysosomal protein usually results in the detrimental accumulation of metabolite. For example, in Hurler's, Hunter's, Morquio's, and Sanfilippo's syndromes, there is an accumulation of mucopolysaccharides; in Tay-Sachs', Gaucher's, Krabbe's, Niemann-Pick's, and Fabry's syndrome, there is an accumulation of sphingolipids; and in fucosidosis and mannosidosis, there is an accumulation of fucose-containing sphingolipids and glycoprotein fragments, and of mannose-containing oligosaccharides, respectively.

The Golgi apparatus is responsible for addition of post-translational modifications and sorting of cellular proteins to the appropriate organelles within the cell. Many of the molecules that reach the Golgi will be exported out of the cell. In order to route molecules properly, the Golgi attaches post-translational modifications. To transport, process and ship the molecules, the Golgi has a system of vesicles that transport molecules received from the endoplasmic reticulum (ER) to the cell membrane or specific subcellular organelles. As with many other organelles, the Golgi may vary from cell to cell. In many cells there is a single Golgi situated to one side of the nucleus. Some other cells have several Golgi apparati appearing as stacks of membranes distributed throughout the cell. The Golgi is most highly developed in cells which are specialized for secretion such as enzyme releasing cells of the digestive tract. The Golgi apparatus has four important roles: 1) modification of complex molecules (such as proteins) by the addition of sugars; 2) sorting of molecules for either, transport out of the cell or incorporation in the cell membrane (the default pathway), 3) sorting of molecules into a regulated secretory pathway: e.g., one used for insulin; and 4) sorting of proteins destined for lysosomes into vesicles directed to late-endosomes.

The Golgi itself is divided into three functionally separate areas: 1) the cis face receives transport vesicles from the smooth ER; 2) the medial Golgi which adds sugars to both lipids and peptides; and 3) the trans-Golgi network which sorts molecules according to their final destination. In mammalian (including human) cells, the lysosomal sorting signal is a sugar modification—mannose-6-phosphate (M6P). The sortase protein that recognizes the M6P signal is a mannose-6-phosphate receptor (MPR), which is present on the inner surface of the trans-Golgi network (TGN) and facilitates their selective transport into lysosomes, wherein the activated enzymes function. In particular, lysosomal proenzymes undergo a variety of posttranslational modifications, including glycosylation and phosphorylation via the 6′ position of a terminal mannose group. Specifically, lysosomal proenzymes are marked by the presence of mannose-6-phosphate, which is recognized by MPR in the TGN. The presence of organelle specific signals allows small transport vesicles containing the appropriate receptor-bound proteins to be pinched off from the trans-Golgi network and targeted to their intracellular destination. See, generally, Kornfeld, 1990.

The low pH of late endosome lumen is often used by the sortases to release lysosomal proenzymes. The sortase is then recycled back to the trans-Golgi via a distinct class of transport vesicle. The released lysosomal proenzymes then flow, via bulk vesicular traffic, from late endosomes to lysosomes.

It is important to note that the cellular sorting mechanism is not 100% efficient. An important insight into transport of extracellular proenzymes to the lysosome came from studying the MPR, which also exists on the plasma membrane of a cell, where it can bind extracellular proteins marked with mannose-6-phosphate and, remarkably, induce their internalization and transport to lysosomes through the endocytic pathway. Thus, lysosomal proenzymes that are secreted may be salvaged from the extracellular space by MPR and recruited to endocytic vesicles that transport the receptor, and bound proenzyme, to late endosomes, the same organelles that receive lysosomal proenzymes from the trans-Golgi. In this manner, extracellularly provided lysosomal proenzymes can be salvaged and transported to lysosomes. This phenomenon underlies the success of enzyme replacement therapy, for example, in the treatment of Gaucher's disease, systemically injected lysosomal proenzymes circulating in the blood are taken up by diseased cells and transported to the defective lysosome to replace the defective proenzyme with active enzymes.

Since the discovery of lysosomal enzyme deficiencies as the primary cause of lysosomal storage disease (“LSD”) (see, e.g., Hers, 1963), attempts have been made to treat patients having lysosomal storage diseases by intravenous administration of the missing enzyme, i.e., enzyme therapy. For lysosomal diseases other than Gaucher's disease, the evidence suggested that enzyme therapy was most effective when the enzyme being administered was phosphorylated at the 6′ position of a mannose side chain group. For glycogenesis type II, this was tested by intravenously administering purified acid α-glucosidase in phosphorylated and unphosphorylated forms to mice and analyzing uptake in muscle tissue. The highest uptake was obtained when mannose 6-phosphate-containing enzyme was used (Van der Ploeg et al., 1991; U.S. Pat. No. 6,118,045).

Enzyme replacement therapy is an established strategy for treatment of several lysosomal storage disorders (LSDs). A lysosomal proenzyme, genetically deficient in the LSD patient, is provided by systemic injection. The enzyme, internalized from the external media by somatic cells, reaches malfunctioning lysosomes where it is activated and performs its normal function.

There are two problems associated with enzyme replacement therapies. First, the cost of purifying functional lysosomal proenzymes in quantities sufficient for therapy is enormous. Second, systemically delivered enzymes do not cross the blood brain barrier and, therefore, are very limited for treatment of CNS symptoms (E. M. Kaye, 2001).

The blood-brain barrier (BBB) resists transport of therapeutic enzymes from the blood and thus does not allow access to malfunctioning cells in the central nervous system. The BBB is a capillary barrier comprising a continuous layer of endothelial cells which are tightly bound. The BBB excludes molecules in the blood from entering the brain on the basis of both molecular weight and lipid solubility, as described in E. A. Neuwelt et al., 1980, and S. I. Rappaport, 1976. For example, the BBB normally excludes molecules with a molecular weight greater than 180 Daltons. In addition, a similar exclusion occurs on the basis of lipid solubility. Thus, cell therapy, the use of cells secreting lysosomal proenzymes inside the brain, is an attractive option for treating neurological symptoms of LSDs.

### SUMMARY OF THE INVENTION

The invention relates to various methods of treating lysosomal storage disorders using somatic cells and methods of delivering therapeutic enzymes. More particularly, the present invention involves the use of gain-of-function or loss-of-function mutations in components of the intracellular Golgi to lysosome sorting pathway as a method by which to enhance secretion of one or more lysosomal enzymes in somatic cells.

The invention also relates to the use of homologous recombination for engineering therapeutically useful cells, for example, somatic cells. The invention further relates to the use of homologous recombination in somatic cells to enhance secretion of one or more lysosomal enzymes.

The invention also relates to the use of homologous recombination for engineering therapeutically useful glial progenitor cells, mesenchymal stem cells, and/or astrocyte precursor cells. The invention further relates to the development of a universal therapeutic glial cell type that can treat lysosomal storage diseases associated with loss of any specific lysosomal enzyme. In one embodiment, a glial progenitor cell is engineered to express a molecule that will, by interfering with the interaction between mannose-6-phosphate and the endogenous MPR or by decreasing the efficiency of cellular sorting, cause increased secretion of M6P targeted proteins, such as, lysosomal proenzymes. In another embodiment, both copies of an endogenous MPR gene are deleted by homologous recombination in the donor cell to cause increased secretion of M6P targeted lysosomal proteins by the donor cell. By secreting multiple proenzymes, not just one specific one, these engineered cells have therapeutic use for many different LSDs.

The invention further relates to transgene expression in cells, including transgene expression in cells produced by homologous recombination. Further, the invention relates to transgene expression in glial progenitor cells, mesenchymal stem cells and/or astrocyte precursor cells.

The invention relates to various methods of treating lysosomal storage disorders using somatic cells and methods of delivering therapeutic enzymes. More particularly, the present invention involves the use of dominant-negative and/or loss-of-function mutations in components of the intracellular Golgi to lysosome sorting pathway as a means to enhance secretion of one or more lysosomal proenzymes in somatic cells. The invention particularly pertains to the use of homologous recombination for engineering therapeutically useful glial progenitor cells, mesenchymal stem cells and/or astrocyte precursor cells expressing a gene product having a gain-of-function, such as a dominant negative, phenotype, but may be easily generalized to include other classes of somatic cells and transgene expression technologies obvious to one skilled in the art.

The invention provides an effective method for treating genetic and/or acquired metabolic CNS disorders, which avoids adverse immunological side effects.

The invention also provides a method for treating genetic and/or acquired metabolic brain disorders which avoids renal clearance problems associated with the direct infusion of purified exogenous enzymes.

The invention further provides a method for treating genetic and/or acquired metabolic brain disorders by providing corrective genetic material to the brain in order to effectively treat the disorder on a molecular level.

The invention also provides a use of the cells and/or methods of the invention for the treatment of metabolic brain and/or CNS disorders. Another aspect of the invention provides the use of the cells and/or methods of the invention for the manufacture of a medicament for the treatment of metabolic brain and/or CNS disorders.

### BRIEF DESCRIPTION OF THE DRAWINGS

FIG. 1 shows an illustration of cell therapy for Lysosomal Storage Diseases.

FIG. 2 shows an illustration of cell therapy for Gaucher's disease.

FIG. 3 shows increased secretion of CPY in a dominant yeast vps mutant (J. Robinson et al., 1988).

FIG. 4 shows two examples of commercial available plasmids for homologous recombination in somatic cells. Note that multiple promoters can be used and the backbone containing the targeting construct can vary.

FIG. 5 shows some of the vector that can be used, wherein the vector is designed to utilize an endogenous promoter, provide ectopic promoters or identify endogenous promoters.

FIG. 6 shows an example of recombination where the gene replaced utilizes the endogenous promoter sequence to drive cell type specific expression.

FIG. 7 shows an example of using a vector containing an IRES site to direct expression of a transcript from an endogenous promoter.

FIG. 8 shows SA sites to disrupt the endogenous gene and generate a desired transcript or to generate a fused transcript.

FIG. 9 shows an example of cell type specific expression with cre mediated recombination to remove the flanking selection sequences.

FIG. 10 shows an example of repeated homologous recombination, wherein repeated targeting can be performed in several ways. One example uses cre/lox mediated recombination sites.

### DETAILED DESCRIPTION OF THE INVENTION

Despite its success for curing many symptoms of specific LSDs, enzyme replacement has not been useful for treating neurological symptoms of LSD (E. M. Kaye, 2001). Two mechanisms for CNS cell therapy are tested in animal models for LSDs. Both mechanisms are strongly influenced by the choice of cell type and suffer from limitations that affect their therapeutic efficiency. The first mechanism involves the transplantation of wild-type cells, which secrete a small amount of lysosomal proenzyme, into the cranial cavity of the subject. Potential problems with this approach include the choice of a cell type that survives and integrates successfully in the human brain. In addition, the range of therapeutic effect provided by a “donor” cell is limited. For example, if lysosomal proenzymes only diffuse 4 cell diameters away from a donor cell, then only localized therapeutic effects can be expected. The second mechanism involves engineering donor cells to over express a specific lysosomal proenzyme that is missing in the specific LSD being targeted for therapy. Because cells engineered to overproduce a lysosomal proenzyme will secrete substantially more enzyme than a wild-type cell, they have the capacity to influence a wider neighborhood of diseased cells. The effective area influenced by the secreted proenzyme is an issue of considerable relevance in transition of therapeutic technologies developed in rodents, which have smaller brains, to humans. However, while cell-based delivery of lysosomal enzymes has been shown to be successful in rodent studies, the techniques for delivery are either not adaptable for humans or have caveats associated with transgene silencing and/or viral transgenesis (U.S. Pat. No. 4,866,042).

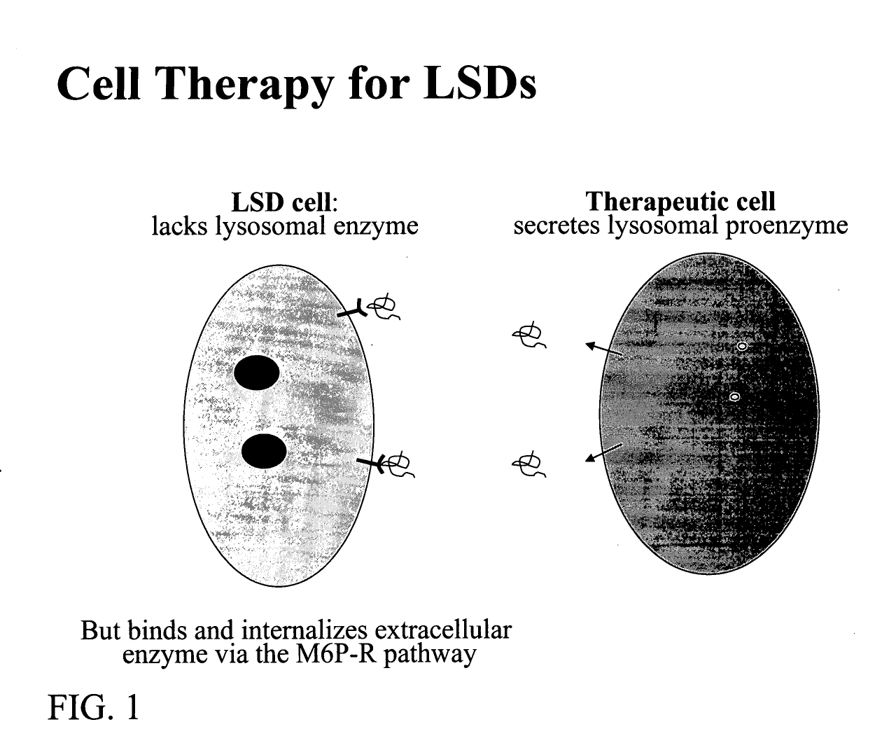

An ideally suited, widely migrating and dividing cell type, such as Cue's Glial progenitor cells, may still have two potential limitations: un-engineered cells may secrete only modest amounts of lysosomal proenzyme; cells engineered to over express a specific enzyme are highly therapeutic for one LSD, but have not been shown to have substantial benefit for other LSDs. Therefore, treatment can benefit from the creation of a class of therapeutic cells that shows increased secretion of all, or a wide spectrum of, lysosomal proenzymes (see, FIG. 1). In the brain, this single cell would be useful to treat neurological deficits in a wide range of LSDs. For non-neurological symptoms associated with LSDs, such a technology could reduce or preclude the need for multiple enzyme injections associated with enzyme replacement therapies. The methods of making and using such a universal LSD therapeutic cell, suggested by analysis of intracellular sorting pathways used for appropriate targeting of lysosomal enzymes, form the core of the invention.

As used herein, “dominant negative” means a mutation that disrupts the function of the wild-type allele in the same cell.

As used herein, “dominant” means that in a diploid organism the phenotype of a dominant gene will manifest in the homozygous or heterozygous state.

As used herein, “proenzyme” and “enzyme” means a protein, or ordered aggregate of proteins, that is capable of catalyzing a specific biochemical reaction, wherein a proenzyme may be subsequently modified, for example, by cleavage of a signal sequence.

As used herein, “frameshift” means a mutation involving a deletion or insertion of a nucleotide that changes the reading frame of the gene. Typically, the stop codon thus formed will not be the normal one, frequently resulting in a truncated or elongated protein.

As used herein, “gain-of-function” means a mutation that produces a new phenotype, including, a hypermorph, a neomorph, an antimorph (e.g., dominant negative) and ectopic expression, which is frequently dominant to wild-type.

As used herein, “gene therapy” means the introduction of nucleic acid into a cell for the purpose of altering the course of a medical condition or disease.

As used herein an “isolated nucleic acid” means a nucleic acid that is not immediately contiguous with both of the coding sequences with which it is immediately contiguous (one on the 5′ end and one on the 3′ end) in the naturally-occurring genome of the organism from which it is derived. The term therefore includes, for example, a recombinant nucleic acid which is incorporated into a vector; into an autonomously replicating plasmid or virus, or into the genomic DNA of a prokaryote or eukaryote, or which exists as a separate molecule (for example, a cDNA or a genomic DNA fragment produced by PCR or restriction endonuclease treatment) independent of other sequences. It also includes a recombinant nucleic acid which is part of a hybrid gene encoding additional polypeptide sequence.

As used herein, “knock out” means the process of introducing a mutation into an endogenous gene to inactivate or reduce the function (or knock out) of the gene.

As used herein, “loss-of-function” means a mutation that reduces or eliminates the function of the gene or gene product, including a null mutation, a hypomorph and a conditional mutation (e.g., temperature sensitive), which are generally recessive to wild-type.

As used herein, “mutation” means any change in the sequence of a nucleic acid relative to wild-type, including insertions, deletions, transitions and transversions of one or more nucleotides. The size of the deletion or insertion can vary from a single nucleotide to many genes. In addition, a mutation means a sequence having a mutation that produces a product capable of producing the mutant phenotype. As known to a person of skill in the art, mutations may display variant degrees of phenotypic penetrance.

As used herein “operably linked” means that the nucleic acid molecule is operably linked to a sequence which directs transcription and/or translation of the nucleic acid molecule.

As used herein “peptide,” “polypeptide” and “protein” include polymers of two or more amino acids of any length. No distinction, based on length, is intended between a peptide, a polypeptide or a protein.

As used herein, “treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

Lysosomal proteins carry a marker, typically, mannose 6-phosphate (M6P). This marker is added exclusively to the N-linked oligosaccharides of the soluble lysosomal enzymes, while they are in the lumen of the cis-Golgi network. The MPR is believed to bind the M6P oligosaccharide in the trans-Golgi network at a pH of about 7, and release it in the late endosome, which is at a pH of about 6. Furthermore, the MPRs have been classified as a cation-independent mannose 6-phosphate receptor (CI-MPR) (GenBank Accession numbers X83699, X83700, X83701 and AFO69333-378; Killian and Jirtle, 1999) or a cation-dependent mannose 6-phosphate receptor (CD-MPR). Both MPRs mediate recruitment of the lysosomal hydrolases within the TGN, from which carrier vesicles deliver the MPR-hydrolase complexes to endosomes. Thus, the lysosomal hydrolases separate from the M6P receptor in the lowered pH of the late endosome and begin to digest the material from the early endosomes.

Because the lysosomal sorting pathway is not 100% efficient, some lysosomal proteins and receptors escape the normal packaging and enter the default pathway to the cell surface. The transport of receptors through the default pathway to the plasma membrane allows these receptors to be used to help rectify errors, such as genetic mutations in one or more lysosomal hydrolase, by capturing extracellular hydrolases through receptor-mediated endocytosis and retargeting to the lysosomes. In brain cells, it has been estimated that a very small amount of lysosomal enzyme (estimated at 0.01% of normal levels) can provide substantial therapeutic effect.

Retrograde transport occurs between the plasma membrane and the endoplasmic reticulum via endosomes and the trans-Golgi network, with an alternative recycling pathway between the Golgi apparatus and the endoplasmic reticulum that is independent from the KDEL receptor (Girod et al., 1999; White et al., 1999).

After releasing their bound enzymes, the receptors are recycled through vesicles derived from buds in the late endosomes and returned to the membrane of the trans-Golgi network. Sorting of MPRs from the TGN to endosomes is mediated by signals present in the cytosolic tails of the receptors. These signals consist of a cluster of acidic amino acid residues followed by two leucine residues. This signal toward the carboxy terminus of the cytoplasmic tail of the protein is necessary for efficient sorting to the lysosome. Mutations in this acidic cluster-di-leucine motif result in increased secretion of lysosomal enzymes. Thus, the mutations in the acid cluster di-leucine motif represent an embodiment of the invention.

Clathrin-associated proteins, including AP-1, are believed to be responsible for the signal-mediated sorting of lysosomal proteins at the TGN. However, clathrin is not required for transport of the MPRs from the late endosome to the TGN. In contrast, clathrin is required for proper localization of molecules having signals such as the tyrosine-based sorting signal of H2M or the di-leucine-based sorting signal of CD3γ. The Golgi-localized, γ-ear-containing, ARF-binding proteins (GGAs) are believed to function in this role. Three GGAs have been identified in humans (GGA1, GGA2, and GGA3) and two in yeast (Gga1p and Gga2p). These proteins are monomeric and display a modular structure consisting of a VHS (VPS27, Hrs, and STAM) domain of unknown function, a GAT domain that interacts with the guanosine 5′-triphosphate-bound form of ADP-ribosylation factors (ARF), a hinge domain that interacts with clathrin, and a GAE domain that interacts with γ-synergin and other potential regulators of coat assembly. Disruption of the two yeast genes encoding GGAs results in impaired sorting of pro-carboxypeptidase Y to the vacuole, the equivalent of the mammalian lysosome. MPR interacts with the ˜150-amino acid VHS domain of all three GAAs (R. Puertollano et al., 2001). Thus, changes in one or more GGA proteins, for example, increased expression or dominant mutations, represent embodiments of the invention.

AP-1 is generally important for TGN to endosome and endosome to TGN transport, for example, the signal YxxF, and the di-Leucine motif bind to AP-1. In addition to AP-1, AP-2 is generally important for endocytosis, AP-3 is generally important for TGN to lysosome transport, AP-4 is known, but the function remains unknown. Not all AP complexes are linked to clathrin, for example, AP-3 may not use Clathrin. Many lysosomal membrane proteins are never found in early endosomes, these lysosomal membrane proteins often require AP-3 subunits, not AP-1. Subtle differences in the YXXF motif determine differences between AP-3 and AP-1 requirements. AP-3 is also important for lysosomal related structures like melanin granules.

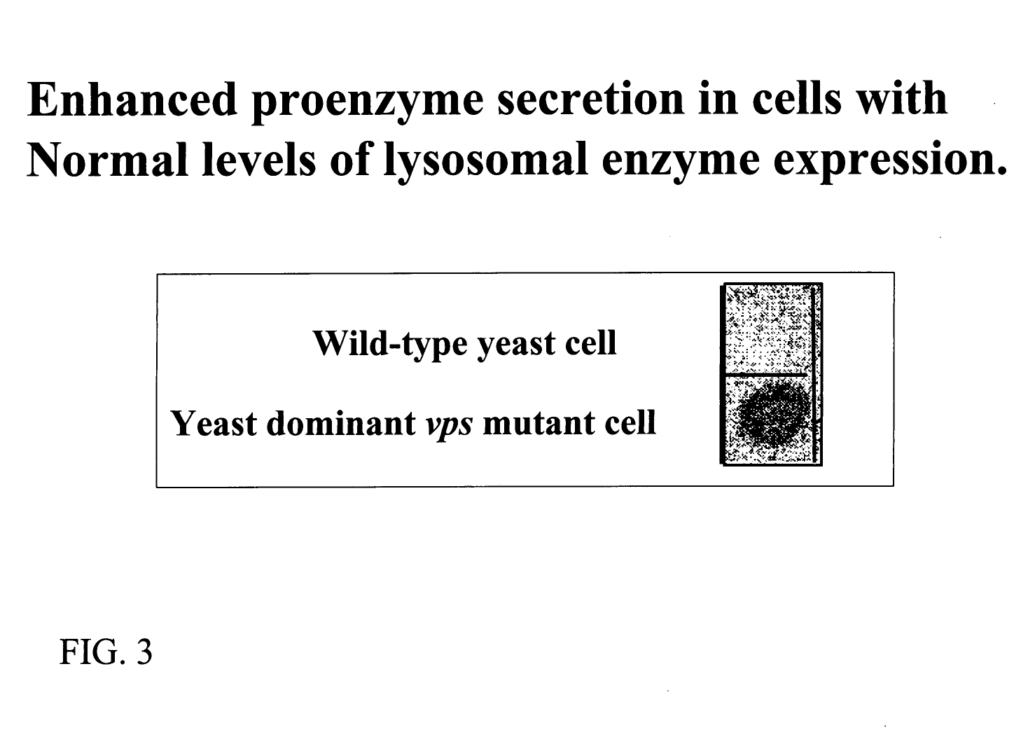

A number of lysosomal sorting genes are known, including genes identified in Saccharomyces cerevisiae such as VPS1 through 6, VPS8 through 11, VPS 13, VPS15 through 30, VPS32 through 39, VPS 41, VPS43 through 45, VPS52 through 55 and VPS60 through 75 (see, FIG. 3). The nucleic acid sequence, amino acid sequence and function of these genes may be obtained from the Saccharomyces Genome Database (SGD) available at http://www.yeastgenome.org, which is incorporated by reference. The function of these gene products is believed to be conserved in metazoan cells (yeast to human). In particular, one of the more divergent members is the vacuolar sortase Vps10p, which in yeast recognizes a peptide signal rather than the M6P signal used by the MPR in mammalian cells, however, Vps10p function is otherwise similar to the function of the human orthologue. In addition, lysosomal sorting genes (orthologues, paralogues and homologues) are known in other organisms, for example, SKD1 is a mouse orthologue of yeast Vps4p (Yoshimori et al., 2000). In addition, gene products, such as, Dynamin, Rab1, Rab7, Rab9, GGAs 1-3, AP-1, AP-2, clathrin a large family of Eph tyrosine kinase (TK) receptors and their membrane bound ephrin ligands are known to play a role in cellular trafficking, for reviews see (Cowan and Henkemeyer, 2002; Kullander and Klein, 2002). Furthermore, gene products known to function between the TGN and endosome, such as Class C mutants, include the GGAs, Rab9, VPS5, VPS17, VPS29 and VPS35.

The invention further relates to dominant and dominant-negative mutations in genes involved in the cellular sorting pathway. Dominant and dominant-negative mutations are known for genes and gene products, such as, vps1, vps4 and vps6/pep12. In addition dominant-negative forms can be created by methods known in the art, such as, a) expressing specific domains of multidomain proteins (as for vps1), b) mutating a domain to alter its activity (e.g., mutating the active sites of an enzyme to decrease function) and c) over expressing one or more members of a pathway (such as Vps4p). The constructs may be maintained as an extrachromosomal sequence or may be introduced into the genome by homologous recombination.

A mutation (e.g., loss-of-function, gain-of-function, dominant or dominant negative), identified in one orthologue may be used to produce a corresponding mutation in other orthologues, paralogues or homologues. Corresponding dominant negative mutations may be introduced into analogous positions of an orthologue, for example, the dominant negative mutations identified in Saccharomyces Vps4p, (E233Q), (E211K) and (G178D), may be used to generate dominant negative mutations in orthologous genes. For example, mouse SKD1 (E235Q) has been shown to be equivalent to the yeast vps4 (E233Q) mutant, wherein both exert an effect on the intra-Golgi transport in vitro (Yoshimori et al., 2000). Likewise, human VPS4A (E228Q) and VPS4B (E235Q) exhibit a dominant negative phenotype similar to the dominant negative mutation in yeast VPS4 (E233Q), when expressed in yeast cells (Scheuring et al., 2000). Both Vps₄p^{E233Q} and SKD1^{E325Q} are inactive in ATP hydrolysis. A dominant negative mutant can be expressed (or over-expressed) to produce the mutant phenotype in otherwise wild-type cells. Id. In addition, over expression of full length clones may also induce a mutant phenotype, presumably by disrupting the balance between the over expressed gene product and other cellular products. Another means for inducing a dominant mutant phenotype is by expression of truncated forms of a gene product. For example, a mutant phenotype may be generated by expression of a dominant-negative form of clathrin, termed the hub fragment (the carboxy-terminal third of the clathrin heavy chain) (Liu et al., 1998).

The use of promoters, known in the art (e.g., the tet-system, T7, Sp6, etc.), allows for the regulation of genes operably linked thereto. Thus, expression of constructs in human cells may be controlled so as to produce a dominant-negative effect. DNA constructs of the invention may also be expressed using promoters of the modified genes or other constitutive, inducible or regulatable promoters. Examples of promoters include, but are not limited to: viral promoters; a neuron-specific enolase promoter (Andersen et al., 1993; Alouani et al., 1992); a MAP-1B promoter (Liu and Fischer, 1996); an L1 promoter (Chalepakis et al., 1994); an aromatic amino acid decarboxylase promoter (Le Van Thai et al., 1993); a dopamine β-hydroxylase promoter (Mercer et al., 1991); an NCAM promoter (Holst et al., 1994); an HES-5 HLH protein promoter (Takebayashi et al., 1995); a α1-tubulin promoter (Gloster et al., 1994); a peripherin promoter (Karpov et al., 1992); a synapsin promoter (Chin et al., 1994); a GAP-43 promoter (Starr et al., 1994); a cyclic nucleotide phosphorylase I promoter (Scherer et al., 1994); a myelin basic protein promoter (Wrabetz et al., 1993); a JC virus minimal core promoter (Krebs et al., 1995); a proteolipid protein promoter (Cambi and Kamholz, 1994); and a cyclic nucleotide phosphorylase II promoter (Scherer et al., 1994) (see, U.S. Pat. No. 6,245,564).

Glial based delivery of lysosomal proenzymes, for example, by integration at a locus actively transcribed in glia (e.g., Rosa, Polr2a) of an expression construct expressing a dominant negative gene product, which results in enhanced secretion of lysosomal proenzyme by the donor cell, is ideally suited for treating lysosomal storage diseases that affect the CNS.

In epithelial cells and/or neurons, a need frequently exists to transport various components to a specific plasma membrane, for example the apical or basolateral membrane. The basolateral signal is tyrosine-based, probably specific t subunit. The apical signal is frequently a combination of GPI linkage, rafts (association with lipids that have segregated in a membrane to form a “lipid raft”). Association with particular motors may be critical for efficient transport to a pre-determined plasma membrane surface. In particular, axons and dendrites have many properties that are analogous to apical and basal transport. There are believed to be numerous motors that associate with specific complexes (e.g., NMDA receptors, AMPA receptors, Synaptic vesicle precursors and active zone precursors). Many of these are large complexes (e.g., NMDA receptor complex) that take up all or a large part of the space in any one vesicle, which may decrease budding specificity and allow attachment to a specific motor to function as the transport destination determinant.

Another aspect of vesicle transport is the proper incorporation of molecules called SNAREs. The SNAREs play important roles in membrane fusion and vesicle docking. Interaction between vesicle-associated snares (v-snare) and target membrane-associated snares (t-snare) are important in vesicle transport and vesicle fusion. The v-snare and t-snare form a high affinity SNARE complex that is activated by a-SNAP/NSF (Von Mollard et al., 1997; Söllner et al., 1993).

Cells may be engineered to express an individual lysosomal proenzyme. Such an engineered cell is useful for the treatment of the corresponding LSD. However, such an engineered cell is also useful for the treatment of other LSDs, wherein over expression of the individual lysosomal proenzyme results in increased secretion of other lysosomal proenzymes, due to overloading of the cellular trafficking system and the resultant use of the default secretory pathway. Alternatively, a universal therapeutic cell type, for example, a glial cell, is created by introduction of a heterologous sequence, which may include gain-of-function or loss-of function mutations, which interfere with normal cellular trafficking, thereby resulting in more lysosomal proenzymes being shunted into the default secretion pathway. In particular, glial progenitor cells are modified to express a molecule that interferes with the interaction between mannose-6-phosphate and the endogenous MPR, causing highly increased secretion of lysosomally targeted proenzymes (see, U.S. Patent Application Ser. No. 60/440,152, incorporated herein by reference). In addition, a cell modified so as to increase secretion of proenzymes, not just one specific proenzyme, results in a donor cell that has therapeutic use for many different LSDs.

One key to successful homologous recombination in stem or self-renewing progenitor cells (primary cells) is achieving the ability to propagate these cells essentially unchanged in culture for many generations. This ability may be assayed directly by passaging the cells in culture for many generations or inferred from high expression levels of the enzyme telomerase that marks stem, progenitor and transformed cells. It was recently shown that glial progenitor cells may be maintained through more than 30 generations in culture and express high levels of telomerase. Mesenchymal cells may also be propagated for more than 40 generations in culture and exhibit high telomerase levels. Other classes of stem and progenitor cells are expected to exhibit similar characteristics including, but not limited to astrocyte precursor cells. See, Sommer and Rao, 2002; Rao et al., 1998; and Rao and Mayer-Proschel, 1997.

Further, glial progenitor cells may be isolated, foreign genes introduced and the cells can be selected for expression of the foreign gene. See, Wu et al., 2002. Further, glial progenitor cells express high telomerase levels. See, Sedivy, 1998. In addition, more than 90% of the CNS cells are glia and are essential for maintaining neuronal survival and normal function, modulating neurotransmitter metabolism, and synthesizing myelin to maintain optimal signal propagation between neurons. Glial dysfunction is also a major factor in neurodegenerative diseases such as lysosomal storage disorders including, but not limited to, Tay-Sachs disease, Hurler syndrome, Gaucher's disease, Fabry's disease and Late Infantile Neuronal Ceroid Lipofuscinoses (“LINCL”). Therefore, glial cells are important in the treatment of the neurological effects of LSDs.

Glial progenitor and astrocyte precursor cells are also ideal therapeutic delivery vehicles because of their exceptional capacity to multiply, migrate and differentiate into oligodendrocyte and astrocyte subtypes. Thus, LSDs may be treated by genetically encoding, for example, glial progenitor cells to express gene products that result in increased secretion of lysosomal proenzymes and delivering the cells to a subject as a part of a cell replacement therapy. Furthermore, the invention demonstrates that homologous recombination occurs efficiently in at least one specific genetic locus in glial progenitor cells, mesenchymal stem cells and astrocyte precursor cells.

The ability of a donor cell to treat different LSDs may be tested by addressing whether the engineered cells can alleviate symptoms in mouse models for different LSDs. Alternatively, coincubation in cell culture may be used to test the engineered cells for the ability to alleviate symptoms of LSDs, wherein the symptoms may be assayed by phenotypic observation or biochemical analysis. Therefore, the cells of the invention, for example, glial progenitor cells, are implanted into a subject to treat LSDs. The cells of the invention may further be used to treat the neurological deficits caused by LSDs, by implantation of the cells behind the blood brain barrier, thereby overcoming the barrier.

DNA may be introduced into a cell by a variety of methods including, but not limited to, electroporation, cell fusion, viral infection, cationic agent transfer, CaPO₄ and transfection. The DNA may be introduced in a variety of forms including, but not limited to, DNA plasmids, lambda phage, BAC (bacterial artificial chromosome), YAC (yeast artificial chromosome), viral vectors (adenovirus vectors, AAV vectors and retroviral vectors) and may be linear or circular. In another embodiment, an internal ribosome entry site (“IRES”) may be inserted into a gene to be integrated at a particular locus where homologous recombination will occur so that the recombined gene will be regulated by an endogenous promoter.

Many different LSDs are known, including the representatives shown in Table 1 and the defective enzyme associated with the disease.

TABLE 1

| Disease | Enzymatic Defect |
|---|---|
| Pompe disease | acid α-glucosidase (acid maltase) |
| Hurler disease* | α-L-iduronidase |
| Hunter disease* | iduronate sulfatase |
| Sanfilippo* | heparan N-sulfatase |
| Morquio A* | galactose-6-sulfatase |
| Morquio B* | acid β-galactosidase |
| Sly disease* | β-glucoronidase |
| I-cell disease | N-acetylglucosamine-1-phosphotransferase |
| Schindler disease | α-N-acetylgalactosaminidase |
| | (α-galactosidase B) |
| Wolman disease | acid lipase |
| Cholesterol ester | acid lipase |
| storage disease | |
| Farber disease | lysosomal acid ceramidase |
| Niemann-Pick disease | acid sphingomyelinase |
| Gaucher's disease | β-glucosidase (glucocerebrosidase) |
| Krabbe disease | galactosylceramidase |
| Fabry disease | α-galactosidase A |
| GM1 gangliosidosis | acid β-galactosidase |
| Galactosialidosis | β-galactosidase and neuraminidase |
| Tay-Sach's disease | hexosaminidase A |
| Sandhoff disease | hexosaminidase A and B |
| Neuronal Ceroid | Palmitoyl Protein Thioesterase (PPT) |
| Lipofuscinsosis. (CLN-1) | |
| Neuronal Ceroid | Tripeptidyl Aminopeptidase I1(TPP-I) |
| Lipofuscinsosis. (CLN-2) | |

=mucopolysaccaridosis

Glycogen storage disease type II (GSD II; Pompe disease; acid maltase deficiency) is caused by deficiency of the lysosomal enzyme acid α-glucosidase (acid maltase). Three clinical forms are distinguished: infantile, juvenile and adult. Infantile GSD II has its onset shortly after birth and presents with progressive muscular weakness and cardiac failure. This clinical variant is fatal within the first two years of life. Symptoms in adult and juvenile patients occur later in life, and only skeletal muscles are involved. The patients eventually die due to respiratory insufficiency. Patients may exceptionally survive for more than six decades. There is a good correlation between the severity of the disease and the residual acid α-glucosidase activity, the activity being 10-20% of normal in late onset and less than 2% in early onset forms of the disease (see, Hirschhorn, pp. 2443-2464).

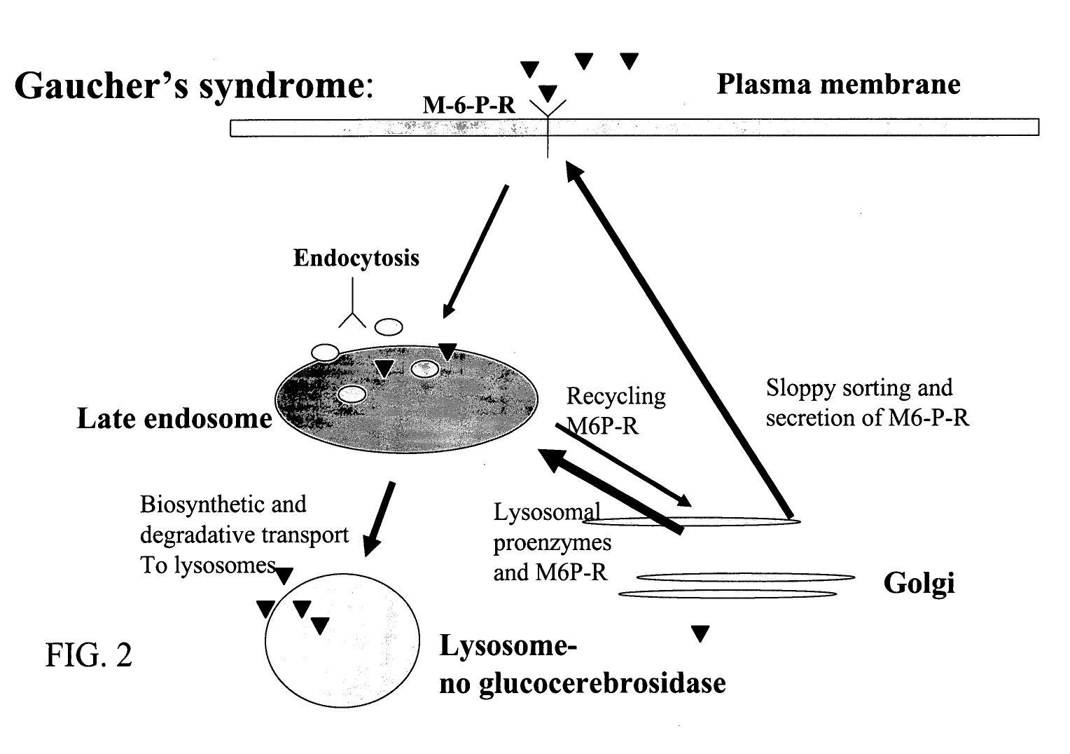

Gaucher's disease is an autosomal recessive lysosomal storage disorder characterized by a deficiency in a lysosomal enzyme, glucocerebrosidase (“GCR”), which hydrolyzes the glycolipid glucocerebroside. In Gaucher's disease, deficiency in the degradative enzyme causes the glycolipid glucocerebroside, which arises primarily from degradation of glucosphingolipids from membranes of white blood cells and senescent red blood cells, to accumulate in large quantities in the lysosome of phagocytic cells, mainly in the liver, spleen and bone marrow. Clinical manifestations of the disease include splenomegaly, hepatomegaly, skeletal disorders, thrombocytopenia and anemia. For example, see U.S. Pat. No. 6,451,600. The present invention provides a therapy for Gaucher's Disease through increased secretion and/or endocytosis of M6P marked GCR, which can correct the enzymatic defect in cells by clearing the stored substrates (see, FIG. 2).

Tay-Sachs disease is a fatal hereditary disorder of lipid metabolism characterized especially in CNS tissue due to deficiency of the A (acidic) isozyme of β-hexosaminidase. Mutations in the HEXA gene, which encodes the α subunit of β-hexosaminidase, cause the A isozyme deficiency. Tay-Sachs disease is a prototype of a group of disorders, the GM2 gangliosidosis, characterized by defective GM2 ganglioside degradation. The GM2 ganglioside (monosialylated ganglioside 2) accumulates in the neurons beginning in the fetus. GM1 gangliosidosis is caused by a deficiency of β-galactosidase, which results in lysosomal storage of GM1 ganglioside (monosialylated ganglioside 1). Sandhoff disease results from a deficiency of both the A and B (basic) isozymes of β-hexosaminidase. Mutations in the HEXB gene, which encodes the β subunit of β-hexosaminidase, cause the B isozyme deficiency.

The present invention provides a therapy for Tay-Sachs Disease through increased secretion and/or endocytosis of M6P marked gangliosidose, which can correct the enzymatic defect in Tay-Sachs cells by clearing the stored substrates, gangliosides.

Another LSD results from a genetic deficiency of the carbohydrate-cleaving, lysosomal enzyme α-L-iduronidase, which causes mucopolysaccharidosis I (MPS I) (E. F. Neufeld and J. Muenzer, 1989; U.S. Pat. No. 6,426,208). See also “The mucopolysaccharidoses” in The Metabolic Basis of Inherited Disease (C. R. Scriver, A. L. Beaudet, W. S. Sly and D. Valle, Eds.), pp. 1565-1587, McGraw-Hill, New York. In a severe form, MPS I is commonly known as Hurler syndrome and is associated with multiple problems such as mental retardation, clouding of the cornea, coarsened facial features, cardiac disease, respiratory disease, liver and spleen enlargement, hernias, and joint stiffness. Patients suffering from Hurler syndrome usually die before age 10. In an intermediate form known as Hurler-Scheie syndrome, mental function is generally not severely affected, but physical problems may lead to death by the teens or twenties. Scheie syndrome is the mildest form of MPS I and is generally compatible with a normal life span, but joint stiffness, corneal clouding and heart valve disease cause significant problems. The frequency of MPS I is estimated to be 1:100,000 according to a British Columbia survey of all newborns (Lowry et al., 1990) and 1:70,000 according to an Irish study (Nelson, 1990).

The present invention provides a therapy for MPS I through increased secretion and/or endocytosis of M6P marked α-L-iduronidase, which can correct the enzymatic defect, as assayed in culture by clearing the stored substrates, heparan sulfate and dermatan sulfate.

Fabry disease is an X-linked inherited lysosomal storage disease characterized by symptoms such as severe renal impairment, angiokeratomas, and cardiovascular abnormalities, including ventricular enlargement and mitral valve insufficiency (U.S. Pat. No. 6,395,884). The disease also affects the peripheral nervous system, causing episodes of agonizing, burning pain in the extremities. Fabry disease is caused by a deficiency in the enzyme α-galactosidase A (α-gal A), which results in a blockage of the catabolism of neutral glycosphingolipids, and accumulation of the enzyme's substrate, ceramide trihexoside, within cells and in the bloodstream. Due to the X-linked inheritance pattern of the disease, essentially all Fabry disease patients are male. Although a few severely affected female heterozygotes have been observed, female heterozygotes are generally either asymptomatic or have relatively mild symptoms largely limited to a characteristic opacity of the cornea. An atypical variant of Fabry disease, exhibiting low residual α-gal A activity and either very mild symptoms or apparently no other symptoms characteristic of Fabry disease, correlates with left ventricular hypertrophy and cardiac disease (Nakano et al., 1995). It has been speculated that reduction in α-gal A may be the cause of such cardiac abnormalities.

I-cell disease is a fatal lysosomal storage disease caused by the absence of mannose-6-phosphate residues in lysosomal enzymes. N-acetylglucosamine-1-phosphotransferase is necessary for generation of the M6P signal on lysosomal proenzymes. Thus, the invention provides a possible cure for this disease.

The invention may be used to treat Fabry disease by introducing a cell over expressing α-gal A or by introducing a cell having a mutation that decreases the accuracy of subcellular trafficking and that results in the secretion of lysosomal proteins.

Since many, but not all, LSDs are associated with neurological symptoms, it is likely that for those not associated with neurological symptoms, if they are treated by enzyme replacement therapy, allowing prolonged life, neurological deficits may manifest.

A few LSDs are caused by defects in membrane-associated proteins that get to lysosomes by a non-Mannose-6-Phosphate dependent route. Gaucher's protein used in enzyme replacement is generally chemically modified in vitro to have M6Ps so that it can be taken up and transported to lysosomes. When the defective enzyme is a transmembrane protein, secretion and uptake of the enzyme is generally not possible. However, the cells of the invention may be modified to express a soluble form of the defective enzyme, thereby overcoming this limitation.

LSDs which affect the central nervous system require that the replacement enzyme cross the BBB. To accomplish this, the source of the replacement enzyme may be placed within the brain of the subject, thereby bypassing the BBB. Thus, glial progenitor cells are ideal therapeutic delivery vehicles because of their exceptional capacity to multiply, migrate and differentiate into oligodendrocyte and astrocyte subtypes. Thus, LSDs that affect the central nervous system may be treated in a variety of manners, including genetically encoding glial progenitor cells to secrete lysosomal proenzymes, for example, lysosomal proenzymes, and delivering the cells to damaged tissues and/or replacing the defective cells.

The ability of glial progenitor cells to grow in culture, levels of telomerase activity, the ability to divide for prolonged periods in culture and the ability to deliver DNA into the cells using electroporation, Lipofection™ and retroviral infection were evaluated. See, Rao et al., 1998; Rao and Mayer-Proschel, 1997.

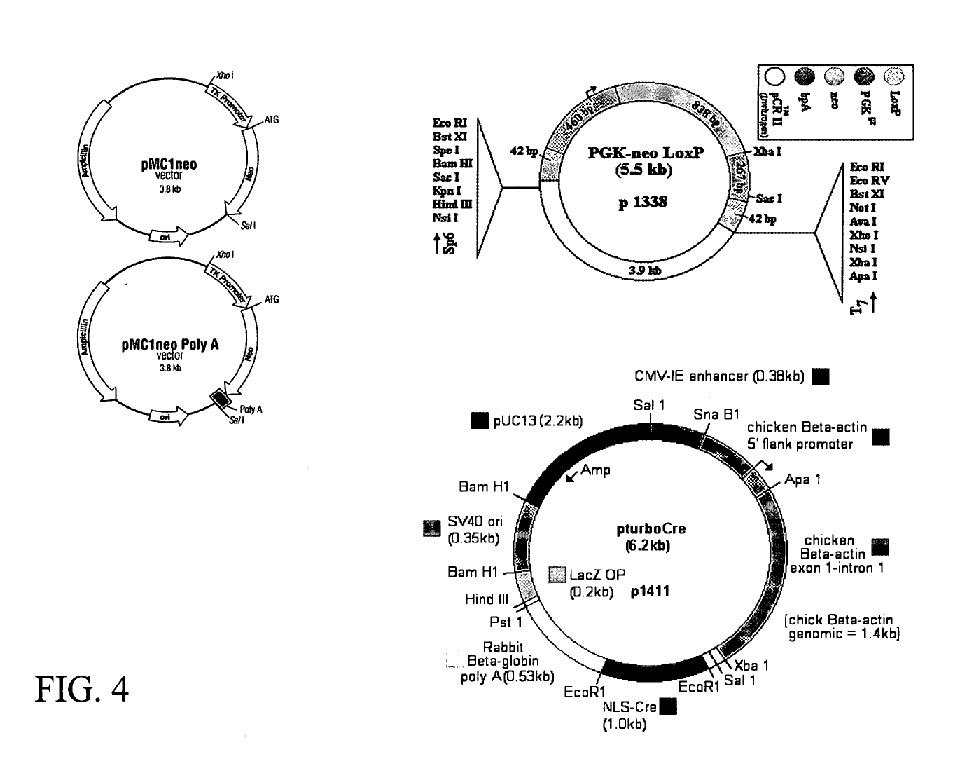

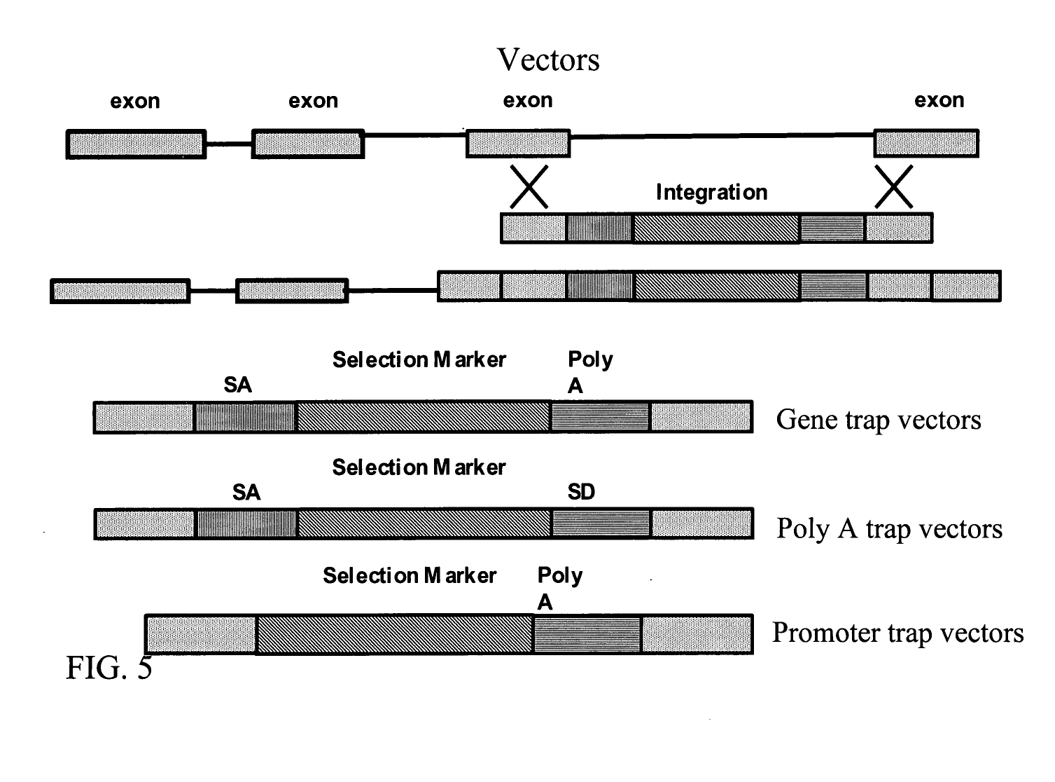

Site specific integration requires the ability to select the cell in which a site specific recombination event has occurred. Primary stem cells constitute an example of a population having a sufficient lifespan in culture to allow for genetic modification, subsequent selection, isolation and cell number expansion. Likewise, other cell types have a sufficient lifespan in culture, for example, glial progenitor, astrocyte precursor, mesenchymal stem cells, embryonic stem cells and embryonic germ cells. We have shown that for glial progenitor cells, astrocyte precursor cells and mesenchymal stem cells, large numbers of cells can be isolated, they self renew, allow transfected genes to be expressed, and are amenable to selection using neomycin and puromycin. For example, electroporation can be used to insert DNA into these cells. In addition, insertion of DNA has been tested using Lipofection™, viral transfer, and calcium phosphate mediated transfer, which suggests that any other standard commercially available gene delivery agent, such as, particle-mediated delivery or microinjection, that has an efficiency of at least 20% may be used according to the present invention. Transformation and transfection methods are described, e.g., in Ausubel et al., supra; expression vehicles may be chosen from those provided, for example, in Cloning Vectors: A Laboratory Manual (P. H. Pouwels et al., 1985, Supp. 1987) or known in the art (see, also FIGS. 4 and 5). For the purposes of ex vivo gene therapy, primary cells are preferred. Primary cells may be grown, transfected, selected, isolated and manipulated by methods known in the art, such as those disclosed in U.S. Patent Application Publication 2002/0012660.

Cells, either prior to manipulation or subsequent to manipulation, may be stored by methods known in the art, such as, freezing the cells in liquid nitrogen, for use at a later time.

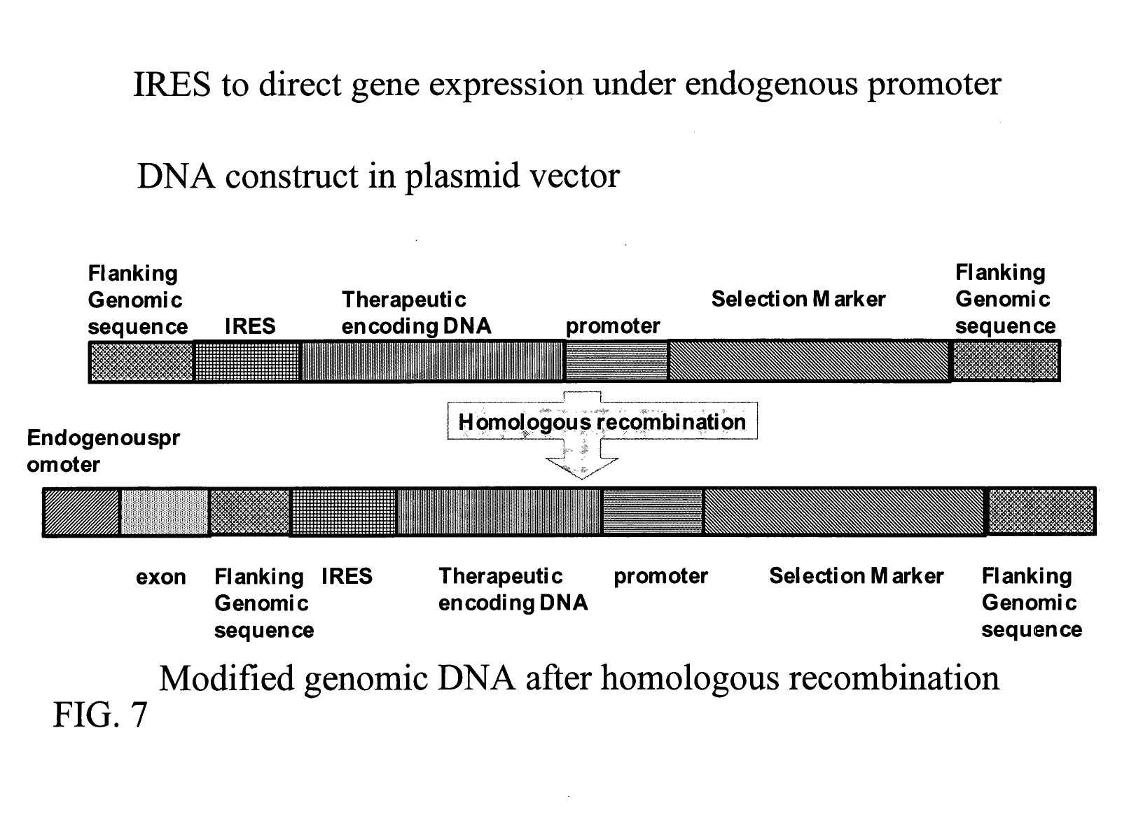

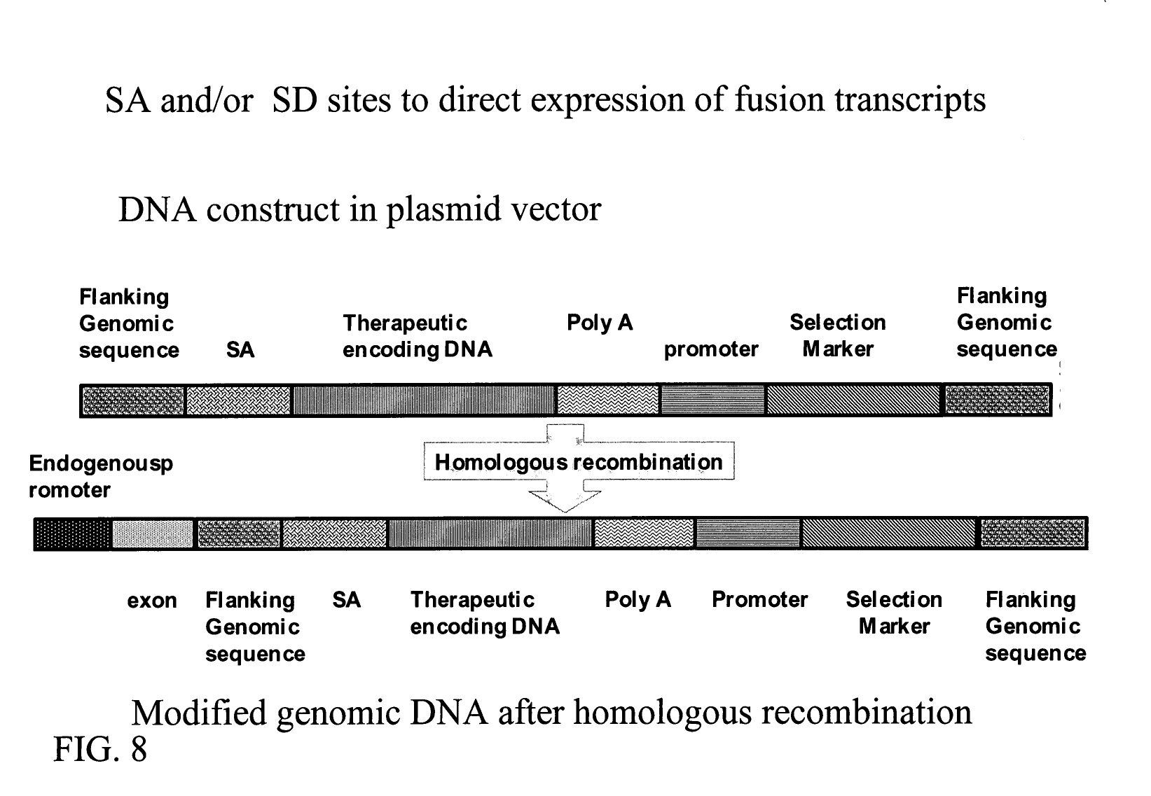

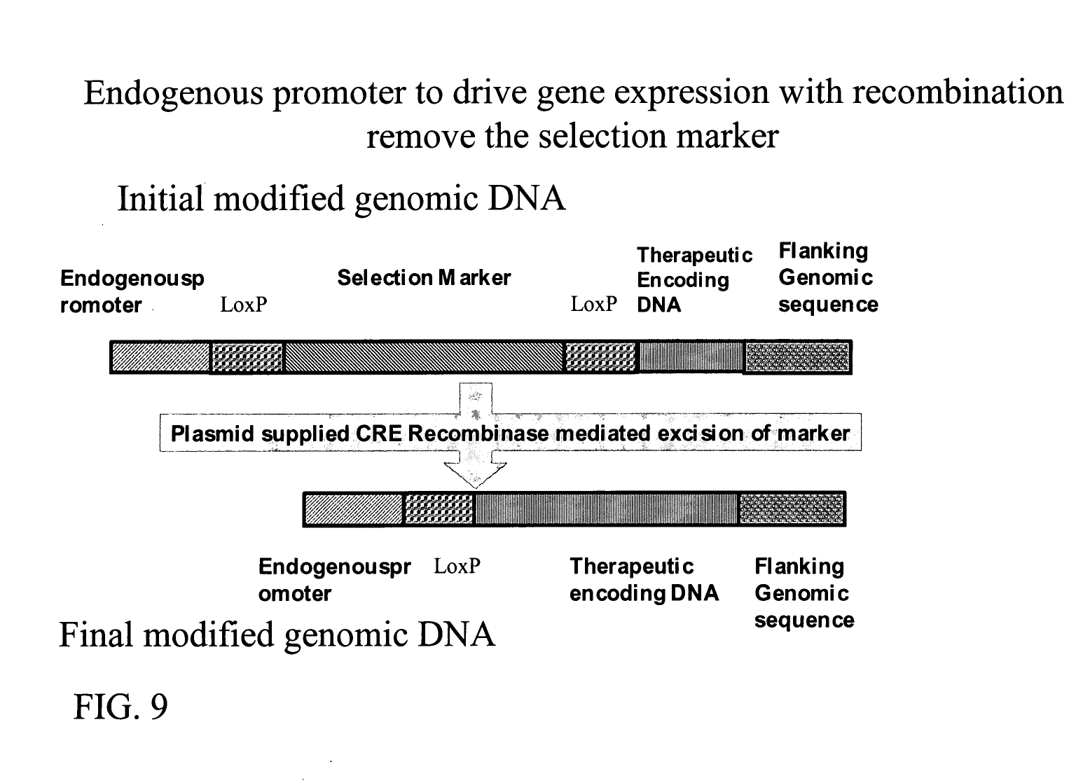

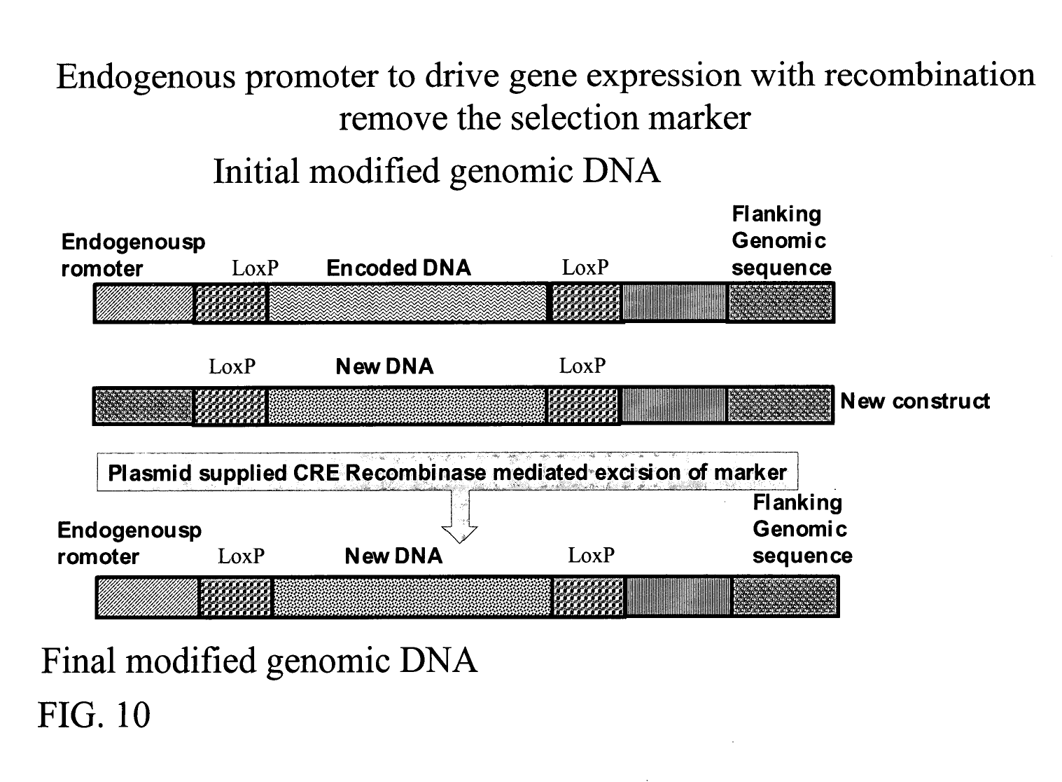

Several constructs, e.g., those that successfully target the Rosa 26 loci, RNA pol II and GAPDH loci, together show that almost any cloned locus of interest can be targeted. Several variations of such plasmids have been used. Furthermore, constructs with internal ribosome entry sites (IRES) sites or cre/lox mediated recombination can be made using methods that are well described and readily obtainable by a person skilled in the art (see, also FIGS. 4 and 7). A detailed review of vectors and constructs used for homologous recombination is described in Court et al., 2002, and Copeland et al., 2001, and examples of some variants of vectors are described herein (see, FIGS. 4 through 11). In particular, FIG. 11 shows use of a highly active, synthetic CAG promoter (H. Niwa et al., 1991) inserted downstream of the Polr2a locus for driving expression of a therapeutic transgene.

Expression vectors may include, for example, an origin of replication or autonomously replicating sequence (ARS) and expression control sequences, such as, a promoter, an enhancer and necessary processing information sites, such as ribosome-binding sites, RNA splice sites, polyadenylation sites, transcriptional terminator sequences, and mRNA stabilizing sequences (see, FIGS. 4 through 10). Signal sequences may also be included where appropriate which allow the protein to cross and/or lodge in cell membranes. Other signals may also be included where appropriate which allow binding to one or more sorting molecules necessary for translocation to a specific cellular compartment (for example, endoplasmic reticulum, nucleus, peroxisome, etc.) and/or retention in a compartment. For example, the amino acid KDEL can be used to retain proteins in the endoplasmic reticulum. Alternatively, when attached to an over expressed protein, the KDEL sequence may be used to swamp the endoplasmic reticulum retention system and increase secretion of such proteins. Such vectors may be prepared by means of standard recombinant techniques well known in the art. See, for example, Ausbel, 1992; Sambrook and Russell, 2001; and U.S. Pat. No. 5,837,492.

### EXAMPLE 1

### Human Vps4 Homologues

The mouse SKD1 is an AAA-type ATPase homologous to the yeast Vps4p, which is implicated in transport from endosomes to the vacuole (Yoshimori et al., 2000). Two human homologues of VPS4 have been identified, VPS4-A and VPS4-B (S. Scheuring et al., 2000). The human VPS4A and VPS4B proteins display a high degree of sequence identity (80%) between them and to the yeast Vps4 protein (59 and 60%, respectively).

VPS4A or VSP4B is amplified by RT-PCR using primers designed by reference to GenBank (accession number AF255952) and/or GeneCard™ accession numbers GC16P060030 (VPS4A) or GC18M060895 (VPS4B) (available through the Weizmann Institute of Science and online at rzpd.de/cards/) and the cDNA product is isolated and cloned into a vector. If the PCR product is less than the full cDNA sequence, 5′ and/or 3′ RACE may be used to obtain the full length cDNA sequence. The sequence of the cDNA present in the vector is verified by sequencing the gene.

### EXAMPLE 2

### Therapeutic Cells Created by Homologous Recombination

A mutation is introduced into the human VPS4A or VPS4B gene to produce a dominant negative point mutation, for example, VPS4A (E228Q) and VPS4B (E235Q), which corresponds to the dominant negative single-point mutation Vps4p (E233Q) that is also equivalent to mouse SKD1 (E235Q) (Yoshimori et al., 2001; Scheuring et al., 2000). The point mutation is introduced by site directed mutagenesis. The dominant negative Vps4Ap^{E228Q}, Vps4Bp^{E235Q}, SKD1^{E235Q} and scVps4p^{E233Q} mutations, all reside within the ATPase module, common to members of the AAA-protein family. Id.

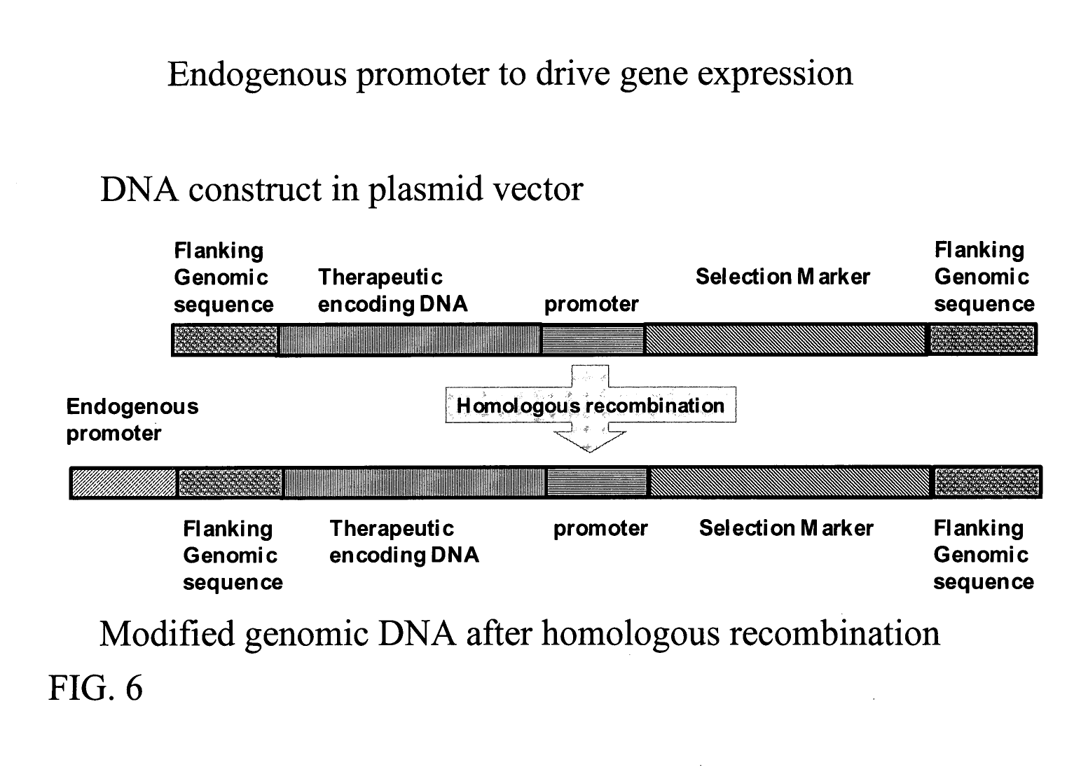

The dominant negative mutant, Vps4Ap^{E228Q} or Vps4Bp^{E235Q}, is cloned into an expression vector, whereby Vps4Ap^{E228Q} or Vps4Bp^{E235Q} is operably linked to a promoter (and optionally an enhancer element) and a poly A signal sequence. The promoter may be the promoter for the endogenous VPS4A or VPS4B genes or may be any appropriate promoter (see, FIGS. 4 through 10). A selectable marker (positive and/or negative selectable marker) is inserted 3′ of the Vps4Ap^{E228Q} or Vps4Bp^{E235Q} gene, for example, Neomycin resistance (alternatively, the gene of interest may function as a selectable or screenable marker) (see, FIG. 6). The expression vector and selectable marker are flanked by genomic sequence 5′ and 3′ of the desired genomic site of integration (see, FIG. 6). The vector is linearized and transfected into host cells, such as glial progenitor cells, mesenchymal stem cells or astrocyte precursor cells. The glial progenitor cells, mesenchymal stem cells and astrocyte precursor cells may optionally be derived from the subject to be treated.

Transformants are identified by selection and stable transformants are further identified. Optionally, stable transformants are tested for expression of the dominant negative mutation and proper integration. For example, expression may be assayed by biochemical identification of the mutant protein and integration may be confirmed by Southern blot analysis or PCR analysis.

The ability of the human mutation to increase secretion of lysosomal proteins may be assayed by methods known in the art. For example, cells expressing Vps4Ap^{E228Q} or Vps4Bp^{E235Q} are incubated with other cells having mutations in a lysosomal hydrolase. The cells lacking the hydrolase are scored for restoration of lysosomal hydrolase activity approximately one to three days post co-incubation. Alternatively, cells expressing hVps4Ap^{E228Q} or hVps4Bp^{E235Q} are cultured in standard media for an appropriate period of time, the media harvested and assayed for the secretion of hydrolase enzyme (proenzyme), secretion is compared to control cells. The presence and/or quantity of hydrolase enzyme in the media may be assayed by ELISA or Western blot assays. Id.

### EXAMPLE 3

### Therapeutic Cells are Expanded and Introduced into a Subject

The engineered cells are expanded and may be exposed to appropriate factors to induce differentiation. A sufficient number of engineered cells are grown and prepared for transplant. A subject, having an LSD, is sedated and the engineered cells are introduced into the subject. For example, the cells may be injected into the spinal chord, cranium or other tissue as appropriate (Kondziolka et al., 2000). The engineered cells introduced into a subject suffering from LSD, thereby treating the disease by secreting a functional proenzyme, which is taken up by the cells of the subject and transported to the lysosome.

### EXAMPLE 4

### Production of a Human MPR Gene Replacement Construct

Two MPRs are known to transport M6P containing proenzymes to the lysosome. The larger of the two receptors has a molecular mass of approximately 300 kDa. This receptor also binds insulin-like growth factor II (IGFII). The larger of the two receptors, MPR/IGF2R (GeneCards™ accession number GC06P159829, available through the Weizmann Institute of Science and online at rzpd.de/cards/, hereby incorporated by reference) may be used according to the invention, however, homozygous loss-of-function has been associated with lethality due to the failure to clear IGFII. The smaller receptor, which has a molecular mass of approximately 46 kDa (the human MPR46 can be found at GeneCard™ accession number GC12M008801, hereby incorporated by reference), is not essential in mice (Köster et al., 1993). MPR46 −/− mice were viable, fertile and lacked an observed phenotype. Id. at 5221. Furthermore, over expression of MPR46 in mice increased secretion of lysosomal proteins from about 10% to about 50% without developing symptoms of a lysosomal storage disorder. Id. Thus, MPR46 provides a gene product which may be used in the invention, since it is not an essential gene product and over expression does not induce a disease phenotype.

The MPRs (for example, Accession number NP000867.1) require TIP47 (also known as PP17, accession numbers O60664; Q9UBD7; Q9UP92), which binds selectively to the cytoplasmic domains of cation-independent and cation-dependent MPRs, for proper sorting.

Human MPR46 is cloned by RT-PCR and inserted into an expression cassette, whereby MPR46 is over expressed. The over expression cassette is introduced into a cassette for homologous recombination. The MPR46 over expression cassette is then integrated into the genome of a host cell, such as, glial progenitor cells, mesenchymal stem cells or astrocyte precursor cells. For example, MPR46 is integrated at the ROSA locus, thereby increasing secretion of lysosomal proenzymes.

### EXAMPLE 5

### Construction of Double Mutant

The construct of Example 4 is combined with a mutation that decreases the availability of MPRs on the surface of the cell over expressing MPR46. A further improvement in the effective amount of secreted lysosomal proteins may be achieved by decreasing the ability of the donor cell to uptake secreted lysosomal proteins. Therefore, the MPR46 over expression cassette of Example 4 is integrated at a genomic site in the host cell and a second mutation is introduced, either concomitantly or subsequently, at the same or a second genomic site. For example, the construct of Example 2 is integrated at a second genomic site is incorporated at the same site, as the MPR46 over expression cassette.

### EXAMPLE 6

### Construction of a Universal Therapeutic Cell

A universal therapeutic cell is created by expressing transgenes encoding a protein that dominantly interferes with normal intracellular sorting mechanisms. For example, a transgene expressing a dominant-mutant form of the mannose-6-phosphate receptor. The MPR is mutated by having mutations in the domains required for sorting into lysosomally directed vesicles, but not in the M6P binding domains, thus, increasing secretion of M6P-containing proenzymes.

Alternatively, a dominant transgene is made in similarly designed variants of known lysosomal sorting proteins such as Rab9 or GGA. In particular, other dominant transgenes are designed based on sequence changes associated with dominant vacuolar sorting mutants in yeast.

Another class of dominant-transgene is produced by over expression of a specific lysosomal proenzyme, thereby saturating the normal intracellular sorting mechanism and increasing secretion of a wide spectrum of lysosomal proenzymes.

The transgene is introduced into a universal cell, for example, a glial progenitor cell, a mesenchymal stem cell or an astrocyte precursor cell. The universal cell may then be further differentiated. The Universal donor cells are sterotactically injectable ventricularly into the parenchyma of a subject.

### EXAMPLE 7

### Expression of a Dominant Negative

A universal therapeutic cell is created by expressing transgenes encoding a protein that dominantly interferes with normal intracellular sorting mechanisms. For example, a transgene expressing a dominant-mutant form of the mannose-6-phosphate receptor. The MPR is mutated by having mutations in the domains required for sorting into lysosomally directed vesicles, but not in the M6P binding domains, thus, increasing secretion of M6P-containing proenzymes.

In Saccharomyces cerevisiae, and mammalian cell culture, enzymes involved in vacuolar protein sorting (vps) have been identified. Mutations in a specific class of vps enzymes (class E vps mutants) have been identified that result in a failure to efficiently sort and/or transport proteins from the early endosome to the lysosome which ultimately leads to mis-sorting to the secretory pathway. One of these enzymes, Vps4, utilizes the energy derived from ATP hydrolysis to disassemble endosome-associated protein complexes that allow multiple rounds of vacuolar protein sorting (Babst et al., 1997). Overexpression of ATPase-defective GFP-Vps4 fusion proteins have been shown to induce formation of enlarged endosomes that inhibit normal recycling of endocytosed substrates resulting in their being mis-sorted to the secretory pathway (Bishop and Woodman, 2000; Garrus et al., 2001).

To create a universal cell line that secretes an unusually high amount of enzymes normally targeted to the lysosome, the effects of transient overexpression of two dominant negative alleles of Vps4 that were fused to GFP (Bishop and Woodman, 2000, 2001) were assayed. One dominant negative Vps4 allele, (Vps4_{K173Q}), blocks ATP binding while allele Vps4_{E228Q} blocks ATP hydrolysis. Vectors expressing GFP alone (vector control) and the GFP-Vps4 dominant negative alleles were transfected into mouse Glial Restricted Progenitor cells (mGRPs). 4×10⁵ mGRP cells were plated in a 6 cm petri dish and grown overnight in DMEM/F12 medium supplemented with N2 supplement (Sigma), B27 (Sigma), bovine serum albumin, fibroblast growth factor, and platelet-derived growth factor and incubated at 37° C. with 5% CO₂. The next day, the cells were transfected with 4 jig of the vector expressing GFP alone, 4 μg of Vps4_{K173Q} and 4 μg VPs4_{E228Q} using FuGene transfection reagents, according to the manufacturer's protocol (Roche). A transfection efficiency of about 30-50% was achieved. The next day, the medium was changed. Two days later, culture medium was collected from each of the plates and proteins precipitated using trichloroacetic acid. The cells were collected by scraping them from the plate and the proteins isolated from the cells using RIPA buffer (1×PBS, 1% Triton X-100, 0.5% sodium deoxycholate, 0.1% SDS). Proteins isolated from the culture medium and the cell lysate were separated onto a SDS-PAGE gel, and blotted to PVDF paper.

Levels of proteins normally targeted to the lysosome are determined by western blot analysis. These proteins include, but are not limited to: cathepsin B, cathepsin D, cathepsin F, cathepsin L, acid ceramidase, and alpha-glucosidase II. Increased levels of these proteins are found in the culture medium from cells transfected with the GFP-Vps4 dominant negative allele plasmids, compared to cells transfected with GFP alone plasmid or untransfected cells. Non-transfected or cells transfected with no DNA or control vector are used as a control.

Alternatively, the level of proteins secreted into culture medium is assayed by determining the activity of enzymes normally targeted to the lysosome. Culture medium from cells transiently transfected with plasmids expressing the GFP-Vps4 dominant negative alleles, a plasmid expressing GFP alone, and untransfected cells are assayed for the presence of alpha-N-acetylglucosaminidase, beta-galactosidase, arylsulfatases A and B, beta-glucuronidase, hexosaminidase, beta-glucosidase and/or alpha-galactosidase using standard methods (Shapira et al., 1989).

While the invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including publications, sequence accession numbers, patents, and patent applications, cited herein are hereby incorporated by reference to the same extent as if each reference were individually and specifically indicated to be incorporated by reference and were set forth in its entirety herein. These references are at least partially set forth in the Bibliography herein.

### BIBLIOGRAPHY

Alouani et al. (1992). Hum. Gene Ther. 3:487-499.
Andersen et al. (1993). Eur. J. Cell Bio. 62:324-332.
Babst M., B. Wendland, E. J. Estepa and S. D. Emr (1998). The Vps4p AAA ATPase regulates membrane association of a Vps protein complex required for normal endosome function. EMBO J. 17, 2982-2993.
Bishop N. and P. Woodman (2000). ATPase-defective mammalian VPS4 localizes to aberrant endosomes and impairs cholesterol-trafficking. Mol. Biol. Cell 11, 227-239.
Bishop N. and P. Woodman (2001). TSG101/mammalian VPS23 and mammalian VPS28 interact directly and are recruited to VPS4-induced endosomes. J. Biol. Chem. 276, 11735.
Cambi F. and J. Kamholz (1994). Transcriptional regulation of the rat PLP promoter in primary cultures of oligodendrocytes. Neurochem. Res. 19, 1055-1060.
Chalepakis et al. (1994). DNA Cell Biol. 13:891-900.
Chin et al. (1994). J. Biol. Chem. 269:18507-18513.
Cloning Vectors: A Laboratory Manual (P. H. Pouwels et al., 1985, Supp. 1987).
Cotran et al., Robbins Pathologic Basis of Disease (4th ed. 1989).
Cowan and Henkemeyer (2002).
Dan R. T. et al. (1976). “Lysosomes,” Essays in Biochemistry 12:1-40.
Garrus J. E., U. K. von Schwedler, O. W. Pomillos, S. G. Morham, K. H. Zavitz, H. E. Wang, D. A. Wettstein, K. M. Stray, M. Cote, R. L. Rich, D. G. Myszka and W. I. Sundquist (2001). Tsg101 and the vacuolar protein sorting pathway are essential for HIV-1 budding. Cell 107, 55-65.
GeneCards™ accession number GC06P159829, GC12M008801, GC16P060030 and GC18M060895 available through the Weizmann Institute of Science and at www.rzpd.de/cards/.
Girod et al. (1999).
Gloster et al. (1994). The T alpha 1 alpha-tubulin Promoter Specifies Gene Expression as a Function of Neuronal Growth and Regeneration in Transgenic Mice. J. Neurosci. 14(12):7319-30.
Hers (1963). Biochem. J. 86, 11-16.
Hirschhorn, The Metabolic and Molecular Bases of Inherited Disease (Scriver et al., eds., 7th ed., McGraw-Hill, 1995), pp. 2443-2464.
Holst et al. (1994). J. Biol. Chem. 269:22245-22252.
Kaye E. M. (2001). Lysosomal Storage Diseases. Current Treatment Options in Neurology 3, 249-256.
Karpov et al. (1992). Biol. Cell 76:43-48.
Killian and Jirtle (1999). Genomic Structure of the Human M6P/IGF2 Receptor, Mammalian Genome 10:7477.
Kondziolka et al. (2000). Transplantation of cultured human neuronal cells for patients with stroke. Neurology 55(4):565-569.
Komfeld (1990). Biochem. Soc. Trans. 18, 367-374.
Köster et al. (1993). Targeted disruption of the Mᵣ 46 000 mannose 6-phosphate receptor gene in mice results in misrouting of lysosomal proteins. EMBO J. 12(13):5219-5223.
Krebs et al. (1995). J. Virol. 69:2434-2442.
Kullander and Klein (2002).
Le Van Thai et al. (1993). Mol. Brain Res. 17:227-238.
Liu and Fischer (1996). Gene 171:307-308.
Liu et al. (1998). A Dominant-negative Clathrin Mutant Differentially Affects Trafficking of Molecules with Distinct Sorting Motifs in the Class II Major Histocompatibility Complex (MHC) Pathway, J. Cell Biol. 140(5):1023-1037.
Lowry et al. (1990). Human Genetics 85:389-390.
Mercer et al. (1991). Neuron 7:703-716.
Nakano et al. (1995). New Engl. J. Med. 333:288-293.
Nelson (1990). Human Genetics 101:355-358.
Neufeld E. F. and J. Muenzer (1989).
Neuwelt E. A. et al. (1980). “Is There A Therapeutic Role For Blood-Brain Barrier Disruption?” Ann. Int. Med. 93:137-139.
Niwa H., K. Yamamura and J. Miyazaki (1991). “Efficient selection for high-expression transfectants with a novel eukaryotic vector,” Gene 108:193-199.
Puertollano R. et al. (2001). Sorting of Mannose 6-Phosphate Receptors Mediated by the GGAs, Science 292:1712-1716.
Rao and Mayer-Proschel (1997). “Glial-Restricted Precursors are Derived from Multipotent Neuroepithelial Stem Cells,” Devel. Biol. 188:48-63.
Rao et al. (1998). “A Tripotential Glial Precursor Cell is Present in the Developing Spinal Cord,” Proc. Natl. Acad. Sci. USA 95:3966-4001.
Rapoport S. I., Blood-Brain Barrier In Physiology And Medicine (Raven Press, N.Y. 1976).
Robinson J., D. Klionsky, L. Banta and S. D. Emr (1988). Protein Sorting In Saccharomyces Cerevisiae: Isolation Of Mutants Defective In The Delivery And Processing Of Multiple Vacuolar Hydrolases, Mol. Cell. Biol. 8:4936-4948.
Robinson J., Ph.D. Thesis, Caltech 1991.
Shapira E., M. G. Blitzer, J. B. Miller and D. K. Africk. Biochemical Genetics. A laboratory manual, 1^{st} ed. New York: Oxford University Press 1989: 1-46.
Scherer et al. (1994). Neuron 12:1363-1375.
Scheuring S. et al. (2000). “Mammalian Cells Express Two VPS4 Proteins Both of Which are Involved in Intracellular Protein Trafficking,” J. Mol. Biol. 00:1-12.
Sedivy (1998). “Can Ends Justify the Means? Telomeres and the Mechanisms of Replicative Senescence and Immoralization in Mammalian Cells,” PNAS USA 95:9078-9081.
Sly and Vogler (2002). “Brain-directed Gene Therapy for Lysosomal Storage Disease: Going Well Beyond the Blood-Brain Barrier,” Proc. Natl. Acad. Sci. 99(9):5760-5762.
Söllner et al. (1993). SNAP Receptors Implicated in Vesicle Targeting and Fusion. Nature 362:318-324.
Sommer and Rao (2002). “Neural Stem cells and Regulation of Cell Number,” Progress in Neurobiology, 66:1-18.
Starr et al. (1994). Brain Res. 638:211-220.
Takebayashi et al. (1995).
The Mucopolysaccharidoses in “The Metabolic Basis of Inherited Disease” (C. R. Scriver, A. L. Beaudet, W. S. Sly and D. Valle, Eds.), pp. 1565-1587, McGraw-Hill, New York.
U.S. Patent Application 2002/0012660.
U.S. Patent Application Ser. No. 60/440,152.
U.S. Pat. Nos. 6,426,208, 6,451,600, 6,245,564, 6,118,045, 4,866,042, 6,395,884 and 5,837,492.
Van der Ploeg et al., Pediat. Res. 28, 344-347 (1990); J. Clin. Invest. 87, 513-518 (1991).
Von Mollard et al. (1997). The Yeast v-SNARE Vti1p Mediates Two Vesicle Transport Pathways Through Interactions with the t-SNAREs Sed5p and Pep12p, J. Cell Biol. 137(7):1511-24.
White et al. (1999).
Wrabetz et al. (1993). J. Neurosci Res. 36:455-471.
Winchester et al. (2000). Biochem. Soc. Trans. 28, part 2:150-154.
Wu et al. (2002). “Isolation of a Glial-Restricted Tripotential Cell Line from Embryonic Spinal Cord Cultures,” GLIA 38:65-69.
Yoshimori et al. (2000). The Mouse SKD1, a Homologue of Yeast Vps4p, Is Required for Normal Endosomal Trafficking and Morphology in Mammalian Cells, Mol. Cell Biol. 11:747-763.

1. A cell secreting lysosomal proteins, said cell comprising a somatic cell having increased secretion of at least one lysosomal enzyme or proenzyme.

2. The cell of claim 1, wherein said cell comprises a gain-of-function or a loss-of-function mutation in at least one gene in the intracellular Golgi to lysosome sorting pathway, wherein said mutation results in increased secretion of said at least one lysosomal enzyme or proenzyme.

3. The cell of claim 2, wherein said mutation is introduced into the genome of said cell by homologous recombination.

4. The cell of claim 1, wherein said cell is a glial progenitor cell, a mesenchymal stem cell, or an astrocyte precursor cell.

5. The cell of claim 4, wherein said cell has a dominant negative mutation.

6. The cell of claim 5, wherein said mutation is introduced into the genome of said cell by homologous recombination.

7. The cell of claim 1, wherein said lysosomal enzyme or proenzyme is an M6P targeted protein.

8. The cell of claim 1, wherein said mutation is in an acid cluster dileucine motif of said lysosomal enzyme or proenzyme.

9. The cell of claim 1, wherein a mutation is present in a nucleotide sequence encoding a protein selected from the group consisting of Rab9, a mannose 6 phosphate receptor, MPR46, MPR/IGF2R, TIP47, CI-MPR, CD-MPR, vps, E vps and GGA protein.

10. The cell of claim 5, wherein said dominant negative mutation is a truncated gene product.

11. The cell of claim 2, wherein said mutation is a gain-of-function mutation encoded by a gene operably linked to a heterologous promoter.

12. The cell of claim 1, wherein said mutation is a VPS4A or VSP4B mutation.

13. The cell of claim 5, wherein said cell is a glial progenitor cell that is modified for CNS therapy.

14. The cell of claim 5, wherein said cell is a mesenchymal cell that is modified for non-CNS therapy.

15. A method of treating a subjecting suffering from a disease selected from the group consisting of Pompe disease, Hurler disease, Hunter disease, Sanfilippo, Morquio A, Morquio B, Sly disease, I-cell disease, Schindler disease, Wolman disease, Cholesterol ester storage disease, Farber disease, Niemann-Pick disease, Gaucher disease, Krabbe disease, Fabry disease, GM1 gangliosidosis, Galactosialidosis, Tay-Sach's disease, Sandhoff disease, and Neuronal Ceroid Lipofuscinsosis, said method comprising:
administering to the subject the cell of claim 1, so as to treat the at least one disease.

16. A method of treating the central nervous system in a subject, comprising:
producing a cell, said cell comprising a glial progenitor cell or an astrocyte precursor cell, having an increased secretion of at least one lysosomal enzyme or proenzyme; and
introducing said cell into the spinal chord or central nervous system of a subject.

17. The method according to claim 16, further comprising:
introducing a mutation into the genome of said cell by homologous recombination.

18. The method according to claim 16, wherein said lysosomal enzyme or proenzyme is an M6P targeted protein.

19. The method according to claim 17, further comprising:
introducing a dominant negative mutation operably linked to a promoter, wherein the promoter is capable of expressing said dominant negative mutation in the cell; and
expressing the dominant negative mutation in the cell and increasing secretion of at least one lysosomal enzyme or proenzyme.

20. A method of treating a mammalian subject thought to be suffering from a lysosomal storage disease, the method comprising:
culturing one or more cells, wherein the cell is selected from the group consisting of at least one glial progenitor cell, mesenchymal stem cell, and astrocyte precursor cell;
introducing a nucleic acid sequence of interest into the one or more cells;
recombining by homologous recombination the nucleic acid sequence of interest and a genomic sequence in the one or more cells, wherein at least a part of the nucleic acid sequence of interest produces an increase in secretion of at least one lysosomal enzyme or proenzyme; and
administering the one or more cells to a mammalian subject.

21. The method according to claim 20, further comprising selecting a recombinant cell having at least part of the nucleic acid of stably integrated into the genome of the selected recombinant cell.

22. The method according to claim 21, further comprising culturing the selected recombinant cell and producing an increased number of selected recombinant cells.

23. The method according to claim 1, wherein the subject is a human.
